(19) United States
(12) Patent Application Publication
(10) Pub. No.: US 2006/0292184 A1
(43) Pub. Date: Dec. 28, 2006

(54) Bioadhesive liquid composition which is substantially free of water

(76) Inventors: Johnathan Craig Richardson, York (GB); Colin David Melia, Nottingham (GB); Peter William Dettmar, East Yorkshire (GB); Frank Chadwick Hampson, Hedon (GB); Ian Gordon Jolliffe, Cottingham (GB)

Correspondence Address: FISH & RICHARDSON PC, P.O. BOX 1022, MINNEAPOLIS, MN, 55440-1022, US

(21) Appl. No.: 10/545,974

(22) PCT Filed: Feb. 13, 2004
(86) PCT No.: PCT/GB04/00529

§ 371 Date: Apr. 3, 2006

(30) Foreign Application Priority Data
Feb. 20, 2003 (GB) 0303831.2

(51) Int. Cl. A61K 9/00

(52) U.S. Cl. 424/400

(57) ABSTRACT

A liquid composition for adherence to a bodily surface, notably to a bodily surface, especially a mucosal surface, comprises water-swellable polymer particles suspended in a water-miscible liquid diluent, wherein the liquid diluent is substantially free of water or includes an amount of water insufficient to fully swell the polymer particles. On admixture with water the composition thickens and becomes more adherent to surfaces. Thus the composition may be easy to administer but may become thick and adherent at the treatment site.

The present invention relates to organic compositions. More specifically the present invention relates to liquid compositions capable of thickening in use and/or adhering to a surface; particularly, but not exclusively, to an epidermal or mucosal surface.

Alginate compositions are used in medicine, for example to alleviate the consequences of reflux oesophagitis. However such compositions, whilst of benefit, are not designed to adhere to the mucosal surface of the oesophagus.

U.S. Pat. No. 6,391,294 describes a pharmaceutically acceptable polymeric material formed in situ at a body surface by the reaction of an anionic polymer and a cationic polymer in the presence of water. These polymers may be applied as separate compositions or as a single composition in a non-aqueous carrier, and react together in situ.

International Journal of Pharmaceutics, 238, 2002, 123-132 describes the use of aqueous alginate solutions as a bio adhesive within the oesophagus.

It would be advantageous to provide a composition which is capable of thickening in the region of a target body surface and/or of adhering to same.

In a first aspect of the present invention provides a liquid composition for adherence to a surface, which composition comprises water-swellable polymer particles suspended in a water-miscible liquid diluent, wherein the liquid diluent is substantially free of water or includes an amount of water insufficient to fully swell the polymer particles, wherein the polymer particles do not include both anionic polymer particles and cationic polymer particles.

Although the composition could be used in household fields, it is preferably a composition for adherence to a bodily surface. The water-miscible liquid diluent is preferably a pharmaceutically acceptable diluent. The composition is a therapeutic composition, in preferred embodiments.

The description which now follows is of a composition of the invention intended for therapeutic use. Non-therapeutic applications will be described later. References in the following pages to the nature of the composition - for example to the particulate nature, to the types of diluent which can be used, to suitable anionic polymers which can be used, and so forth—are applicable also to non-therapeutic applications.

The composition of the present invention is thus in the form of a suspension of particles. These particles remain as particles in the composition before it is used, preferably substantially without swelling. They can range widely in size, from visible to the naked eye to microscopic. The suspension may be in form of a homogenous dispersion. The composition may be mixed with water ex-vivo (for example in a glass), for example immediately prior to administration. Alternatively it may be mixed with water in-vivo, for example in the mouth (the saliva providing the water). However delivered, the water causes the polymer particles to swell, allowing them to coalesce, increase the viscosity of the composition and cause at least a proportion of them to adhere to a bodily surface. The particles need not exhibit any dissolution in water but in preferred embodiments they dissolve partially or completely in water. In all embodiments, however, water causes the particles, previously kept in a no- or low-water environment, to swell.

Preferably the adhered coating prevents or alleviates inflammation or damage. It may allow the surface to heal by providing a barrier on top of a damaged surface to protect it from further inflammation or damage.

Alternatively or additionally the adhered coating is such as to promote the absorption, through the bodily surface, of an active pharmacological agent. The active pharmacological agent may be co-formulated with the composition or administered separately. It may be laid down as part of the coating or may be separate, but absorbed through the coating, in use.

A bodily surface could be an epidermal surface. An epidermal surface could be any external surface skin. Damaged skin could be skin which is blistered, burnt by fire, inflamed, pustulated, sunburnt, bitten or stung.

A bodily surface could be a mucosal surface. A mucosal surface could be any internal bodily surface. Examples include the mouth (including tongue), nose, eyes, throat, oesophagus, stomach, vagina and rectum.

A bodily surface could be a torn or cut surface, for example an exposed surface of a muscle, exposed by a wound or other trauma.

A composition of the invention may serve as a skin hydrating or softening composition, or as a hair treatment or hair removing composition.

A composition of the invention may be a dental composition, for example a denture fixative.

When a composition of the present invention is mixed with water in the saliva it is preferably designed to adhere to a surface of the gastro-intestinal tube, preferably to the oesophagus, and most preferably to the lower oesophagus. However, it may be designed to adhere to a different surface, for example a surface of the mouth or throat, for example to relieve mouth ulceration or throat inflammation.

Preferably, the interval between mixing with water and attainment of a beneficial degree of swelling is in the range 1 to 60 seconds, most preferably 2 to 30 seconds.

A suitable polymer is preferably one which is water-swellable, non-toxic and does not swell in the diluent.

Suitably, the polymer may be anionic, cationic or non-ionic. Combinations of such polymers may be employed except that co-formulations of anionic and cationic polymers are not favoured due to interaction between them. Thus the following may suitably be employed as the polymer, in any given formulation:
Anionic polymer(s) only. This is an especially preferred formulation. Within this definition mixed anionic polymers may be employed, but preferably only one anionic polymer is employed.
Non-ionic polymer(s) only. This is a preferred formulation. Within this definition mixed non-ionic polymers may be employed, but preferably only one non-ionic polymer is employed.
Cationic polymer(s) only. This is a preferred formulation. Within this definition mixed cationic polymers may be employed, but preferably only one cationic polymer is employed.
Anionic polymer(s) and non-ionic polymer(s) together. Within this definition mixed anionic polymers and/or mixed non-ionic polymers may be employed, but preferably only one anionic polymer and one non-ionic polymer is employed.
Cationic polymer(s) and non-ionic polymer(s) together. Within this definition mixed cationic polymers and/or mixed non-ionic polymers may be employed, but preferably only one cationic polymer and one non-ionic polymer is employed.

Examples of suitable anionic polymers are given in, for example, U.S. Pat. No. 6,391,294. Preferred anionic polymers include water-soluble salts of hyaluronic acid, salts of alginic acids (e.g. alginates such as salts of alkali and alkaline earth metals, for example sodium alginate, potassium alginate, calcium alginate and magnesium alginate), xanthan gum, acacia, pectins, acidic derivatised polysaccharides preferably uronic acid-containing materials e.g. hyaluronic acids, or sterculia, carrageenan salts and polylactic acids and water-soluble cellulose derivatives (e.g. sodium carboxymethyl cellulose).

More preferred anionic polymers for use in the present invention are water-swellable, preferably water soluble, salts of alginic acids (i.e. alginates) and water-swellable, preferably water soluble, salts of cellulose derivatives.

Example of suitable cationic polymers are given, for example, in U.S. Pat. No. 6,391,294. Preferred cationic polymers include chitosan salts (e.g. chitosan chloride, chitosan acetate), diethylaminoethyl dextran, chondroitin salts, polylysine, dermatan and keratin.

Examples of suitable non-ionic polymers include cellulose derivatives (e.g. methyl cellulose, hydroxyethylpropyl cellulose, hydroxypropylmethyl cellulose, hydroxypropyl cellulose) and starch and starch derivatives.

The polymer particles preferably have, in their unswollen state, a mean particle size of from 30 to 500 micrometers, especially from 50 to 200 micrometers, especially from 90 to 125 micrometers. To measure the mean particle size they may be fractionated by sieving, before preparation of the composition of the invention.

The composition preferably comprises from 2 to 90 wt % of said polymer particles based on the total weight of the composition, more preferably from 5 to 70 wt %, yet more preferably from 20 to 60 wt %, and most preferably from 30 to 50 wt %.

By “water” herein we mean to include aqueous liquids, for example saliva.

The non-aqueous liquid is, of course, itself pharmaceutically acceptable. Preferred non-aqueous liquids comprise or consist of monohydric alcohols, polyhydric alcohols, sugar alcohols and sugar polyols suitable monohydric alcoholics include ethanol and isopropanol.

Suitable polyhydric alcohols include glycerol, glycols, polyalkylene glycols or mixtures thereof. A suitable glycol is, for example, propylene glycol. A suitable polyalkylene glycol is a polyethylene glycol, preferably of molecular weight at least 100, preferably at least 200. Preferably the molecular weight is up to 1,000, more preferably up to 700, most preferably up to 400.

A suitable sugar polyol is hydrogenated glucose syrup (LYCASIN (RTM)).

The pharmaceutically acceptable liquid diluent preferably contains substantially no water, for example less than 1 wt % water, or preferably less than 0.5 wt % water, on total weight of composition. Most preferably is it anhydrous.

Alternatively the pharmaceutically acceptable liquid diluent comprises some water. This may be advantageous in order to tailor the swellability of the anionic polymer particles for optimal efficacy in the required location. For instance, incorporating some water in the composition will cause the particles to swell to a certain extent, but not substantially to coalesce. When a composition comprising partially pre-swelled particles is swallowed and mixed with saliva, the particles will coalesce and form a barrier film quicker than when they are not partially pre-swollen. By analogy they may be regarded as “primed”.

In such embodiments the composition should contain a proportion of water sufficient to “prime” the particles and no more; it is not desired to substantially thicken the composition prior to administration. The optimum proportion of water depends on the other components of the composition, and especially on the liquid diluent. Generally, the liquid diluent may contain 10-70% water, by weight on weight of diluent.

When the liquid diluent is glycerol it may contain up to 20% water, preferably 10-20% water, by weight on weight of diluent (i.e. the glycerol).

When the liquid diluent is a simple glycol, preferably propylene glycol, it may contain up to 50% water, preferably 10-50% water, most preferably 25-50% water, by weight on weight of diluent (i.e. the glycol).

When the liquid diluent is a polyalkylene glycol, for example a polyethylene glycol, it may contain up to 60% water, preferably 10-60% water, most preferably 30-60% water, by weight on weight of diluent (i.e. the polyalkylene glycol). Whilst the upper limit is preferably 60% water by weight of diluent when the diluent is PEG 400, when it is PEG 200 the upper limit is preferably 40%.

Preferably the Hildebrand Solubility Parameter of the diluent (including any water present) is at least 15, preferably at least 20 (Jcm⁻³)^{1/2}.

Preferably the Hildebrand Solubility Parameter of the diluent (including any water present)is not greater than 35, preferably not greater than 31 (Jcm⁻³)^{1/2}.

The composition may also contain an active agent, particularly when the active agent has an effect on an inflamed or damaged bodily surface, for example an oesophagus inflamed by gastric reflux, or when it is desired to permit the active agent to be absorbed into the blood stream through the skin, via the adhered composition. Suitable active agents include analgesics, anti-inflammatory agents and antipyretics (e.g. acetaminophen, ibuprofen, naproxen, diclofenac, ketoprofen, choline salicylate, benzydamine, buprenorphine, hydrocortisone, betamethasone); decongestants (e.g. pseudoephedrine, phenylephrine, oxymetazoline, xylometazoline); mineral salts (e.g. zinc gluconate, zinc acetate); cough suppressants (e.g. dextromethorphan, codeine, pholocodine); expectorants (e.g. guaiphenesin, n-acetylcysteine, bromhexine); antiseptics (e.g. triclosan, chloroxylenol, cetylpyridinium chloride, benzalkonium chloride, amylmetacresol, hexylresourcinol, dichlorobenzyl alcohol, benzyl alcohol, dequalinium chloride, silver sulphadiazine); cardiovascular agents (e.g. glyceryl trinitrate); local anaesthetics (e.g. lignocaine, benzocaine); cytoprotectants (e.g. carbenoxolone, sucralfate, bismuth subsalicylate); antiulcer agents (e.g. calcium carbonate, sodium bicarbonate, magnesium trisilicate, magaldrate, cimetidine, ranitidine, nizatidine, famotidine, omeprazole, pantoprazole); antihistamines (e.g. loratidine, terfenadine, diphenhydramine, chlorpheniramine, triprolidine, acrivastine); antinausea agents (e.g. prochlorperazine, sumatriptan), bowel regulatory agents (e.g. diphenoxylate, loperamide, sennosides); antifungal agents (e.g. clotrimazole); antibiotics (e.g. fusafungine, tyrothricin) and antipsoriasis agents (e.g. dithranol, calcipotriol). One or more agents may be included.

The compositions of the present invention may be intended simply to adhere to a bodily surface in order to treat a condition thereof. However, in the case of oesophageal surface it may additionally function to treat gastrointestinal stress, such as reflux oesophagitis, gastritis, dyspepsia or peptic ulceration. In this aspect of the present invention the composition therefore may also comprise a bicarbonate and optionally an alginate cross-linking agent so that the composition which reaches the stomach will form a reflux inhibiting “raft”. An especially preferred embodiment for such use may comprise a composition of the present invention, together with calcium carbonate and sodium bicarbonate, formulated to be drinkable.

In accordance with a second aspect there is provided a method of treating a patient, using a composition of the invention as defined above, adhered to a bodily surface of the patient. This may be done, for example, in order to prevent or alleviate a medical condition of the bodily surface. Alternatively or additionally it may be done in order to provide an active pharmacological agent to the patient transdermally.

The invention further provides the use of polymer particles in the manufacture of a composition as defined herein, for the treatment of a bodily surface in need of preventative or restorative treatment, or for transdermal delivery of an active pharmacological agent.

The composition of the present invention may be prepared by mixing together the ingredients until a homogeneous mixture, typically a homogeneous dispersion, is achieved.

Non-therapeutic applications of the present invention are applications which also benefit from having initially a composition of low viscosity, and which on dilution with water becomes a liquid of higher viscosity, preferably with a propensity to adhere to a target surface. A composition of the present invention may find application in a household cleaning composition. For example the composition may be used in a device which periodically releases a composition according to the first invention in its non-diluted, non-viscous form, into a lavatory bowl. The composition may run freely down the lavatory bowl into the water, where it thickens and adheres to the sanitaryware below the water line, where it may have a cleaning action. When the polymer is an alginate it may act to prevent or remove limescale, due to the strong seguestrant action of the alginate.

In another non-therapeutic embodiment a composition of the invention may be part of an encapsulated composition for use in a ware-washing machine. The encapsulating material may be water-permeable and the polymer inside the capsule swells as water is taken in, and causes the capsule to rupture, releasing the contents into the ware washing machine. The polymer is then freed and can adhere to the hard surfaces within the ware washing machine. It may thereby function to combat or prevent scale on the surfaces of the ware washing machine.

The invention will now be further described, by way of illustration with reference to the following sets of examples.

### EXAMPLE SET 1

In these examples the aim was to assess the influence of
Diluent dilution by artificial saliva on the rate of alginate particle swelling.
Diluent composition choice on rate of alginate particle swelling.

Materials

| Name | Supplier |
|---|---|
| Protanal LF120L (sodium | FMC BioPolymer AS, Drammen, |
| alginate) | Norway |
| 1,9-dimethylmethylene blue | Sigma-Aldrich Company Ltd, |
| (DMMB) | Dorset, England, UK. |
| Glycerol 99.5% | Sigma-Aldrich |
| Propylene glycol | Sigma-Aldrich |
| PEG 200 | Fluka Chemika |
| PEG 400 | Sigma-Aldrich |

Equipment

Nikon Labophot optical microscope, attached to COHU High Performance CCD Camera interfaced to the image analysis software, Image Proplus v.4.1 (Media Cybernetics, Maryland, USA-Supplier Datacell Ltd, Finchampstead, Berkshire, UK)

Vortex Mixer VM20, Chiltern Scientific, Bucks. UK Thoma Haemocytometer Counting Chamber, Depth 0.1 mm, 0.0025 mm², Hawksley, England.

Methodology

Preparation of Artificial Saliva

Artificial saliva was prepared to the following formula: 5 mM sodium bicarbonate, 7.36 mM sodium chloride, 20 mM potassium chloride, 6.6 mM sodium dihydrogen phosphate monohydrate, 1.5 mM calcium chloride dihydrate in water.

Visualisation of Single Particle Swelling

A single alginate particle (90-125 μm) was placed onto the centre of a haemocytometer counting chamber, covered using a cover slip and the cover slip weighted on either side by Blu-Tack® (Bostick Ltd, Leicester, UK). 15 μL of hydration fluid (artificial saliva: diluent:DMMB) was injected at the front of the chamber close to the cover slip. Capillary forces between the chamber surface and cover slip sucked the fluid between the interface and immersed the single particle. As the alginate particle was s hydrated the gel layer could be delineated due to a colour change from blue to purple upon complexation between DMMB and soluble alginate. Using optical microscopy (Nikon Labophot) the colour change meant it was possible to visualise in two dimensions the radial swelling of the particle. Image analysis software (Image Pro Plus v.4.0, Media Cybernetics, USA) captured an image after a pre-determined time period and the extent of radial particle swell was calculated by software measurements of the swollen area.

The rationale for using the haemocytometer counting chamber was to ensure a fixed volume of swell. The distance between the coverslip and chamber surface is precision engineered to 100 μm therefore alginate particles from the sieve fraction 90-125 μm would be trapped, restricting axial swell. Consequently, swelling will occur radially and the extent of swelling can be calculated from a 2D image using image analysis.

Preparation of Hydration Fluid

The hydration fluid was used to hydrate the alginate particle within the haemocytometer chamber. To determine the influence of diluent choice and dilution of diluent with artificial saliva on particle swelling a range of diluent:artificial saliva solutions were prepared (0-100% w/w). The following diluents were examined:
Glycerol
70:30 w/w glycerol:propylene glycol
40:60 w/w glycerol:propylene glycol
Propylene glycol
PEG200
PEG400

1,9-dimethyl methylene blue (DMMB) was added to the hydration fluid (diluent: artificial saliva mixture). DMMB is a cationic dye that complexes solubilised sodium alginate and the resultant colour change from blue to purple delineates the gel layer of the swollen particle. The concentration of diluent within the hydration fluid determined the amount of DMMB added (350-1490 μM). The amount of DMMB added was the minimum concentration necessary for visualisation.

Results and Discussions

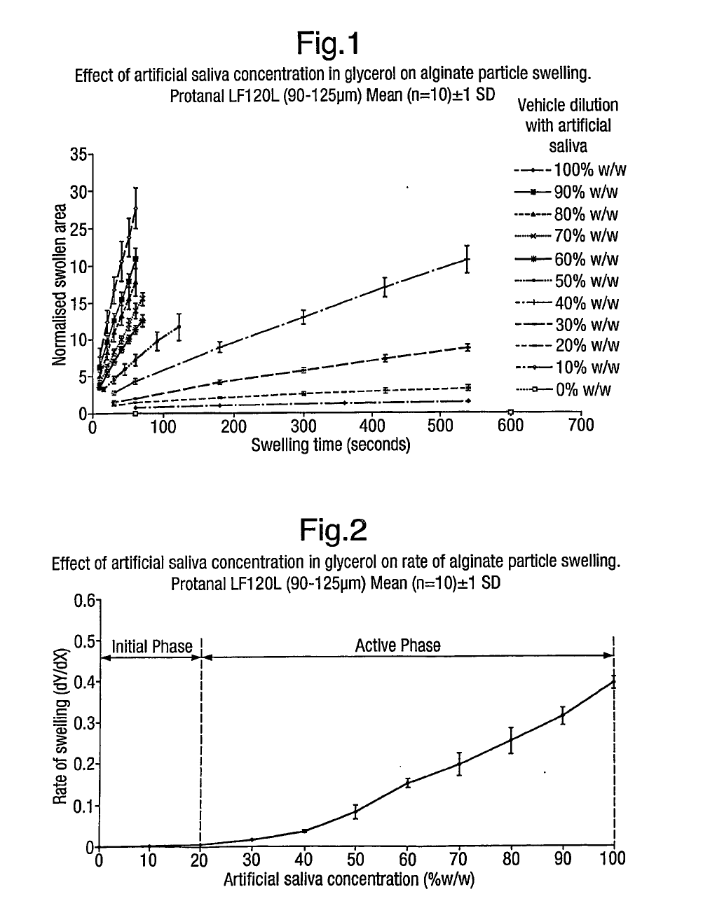

FIG. 1 shows the swelling of single alginate particles in glycerol and illustrates how the swelling behaviour changed upon dilution with artificial saliva. In 100% diluent (i.e. 0% w/w diluent dilution) alginate particles did not swell, as over time there was no change in particle area. However as increasing dilutions of glycerol with artificial saliva were used to hydrate the alginate particle, the rate of swelling, calculated from the gradient of normalized area vs time, increased (FIGS. 1 and 2).

It appears from the results shown in FIGS. 1 and 2 that the relationship between rate of alginate particle swelling and increasing dilution of glycerol with artificial saliva can be considered as having two principal features. Firstly the relationship can be characterised in terms of an initial phase. The initial phase represents a series of diluent dilutions during which the suspended alginate particles remain in the unswollen state. Secondly, at a critical level of dilution the initial phase is exceeded and there is an increase in the rate of swelling with subsequent dilution (active phase).

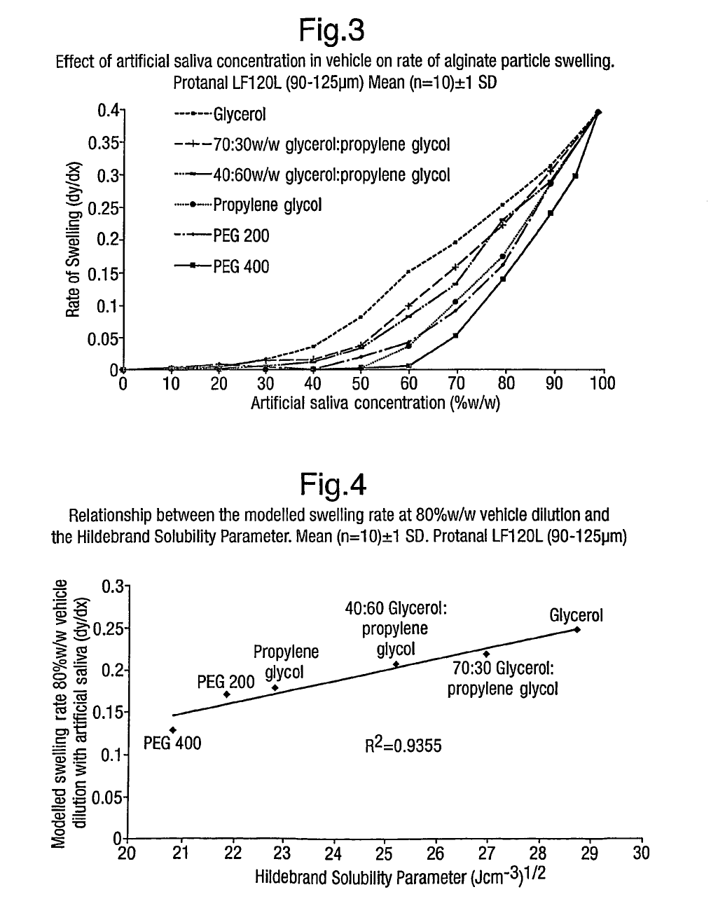

FIG. 3 illustrates that the relationship between rate of swelling and diluent dilution was individual to each diluent.

Different diluents exhibited differences in both the level of dilution necessary to exceed the initial phase i.e. diluent dilution necessary to initiate particle swelling and upon entering the active phase the sensitivity of the rate of swelling to further dilution. It was clearly visible that alginate particles began to swell in approximately 25% w/w glycerol:artificial saliva. However in PEG 400 it was necessary to dilute the diluent by 60% w/w using artificial saliva to induce swelling.

The six diluents offer a range of swelling rates upon dilution, and provide a means of controlling the extent of diluent dilution necessary to activate swelling.

The enthalpy of vaporisation is the amount of energy required to convert a pure liquid to a gas. In converting a liquid to a gas it is necessary to totally separate the individual molecules of the liquid, therefore the enthalpy of vaporisation is a direct measure of the amount of van der Waals forces holding the liquid molecules together.

During the mixing of a solvent and solute, the solute must. disrupt the van der Waal's interactions between the solvent molecules in a manner analogous to vaporization. Consequently, a solvent's enthalpy of vaporisation, which is a measure of the strength of van der Waals interaction between solvent molecules, can provide an indication of solvency behaviour. The solvency behaviour of a particular solvent can be expressed by the Hildebrand Solubility Parameter (δ) which is calculated from the square root of the cohesive energy density.
Hildebrand Solubility Parameter (δ)=(ΔE/V)^{0.5}
(ΔE/V)=Cohesive energy density

Therefore, a solvent's Hildebrand solubility parameter gives a measure of the van der Waals forces between solvent molecules and can be used to rank the solvency behaviour of a range of solvents.

Within the context of this work the solvents used to swell alginate particles were the 6 diluents:
Glycerol
Propylene glycol
70:30 w/w mixture of glycerol:propylene glycol
40:60 w/w mixture of glycerol:propylene glycol
PEG200
PEG400

The Hildebrand solubility parameter of the diluent is believed to be of significance in the present invention. This may be calculated using the group contribution method (refs: Sperling, L. H.< Introduction to Physical Polymer Science, 3^{rd} ED 2001, Wiley; Cowie J. M. G, Polymer Chemists and Physics of Modern Materials, 2^{nd} ed 1991, Glasgow:Blackie)

The Hildebrand solubility parameter was calculated for each diluent as shown in the following table.

| | Hildebrand solubility |
|---|---|
| Diluent | parameter (Jcm⁻³)^{1/2} |
| Glycerol | 28.77 |
| 70:30 w/w glycerol:propylene | 26.99 |
| glycol | |
| 40:60 w/w glycerol:propylene | 25.22 |
| glycol | |
| propylene glycol | 22.81 |
| PEG 200 | 21.83 |
| PEG 400 | 20.81 |

The Hildebrand solubility parameter for each diluent provides a measure of the cohesive forces between the individual diluent molecules, and it appears to be possible to use the Hildebrand solubility parameter to understand the relationship between rate of particle swelling and extent of vehicle dilution.

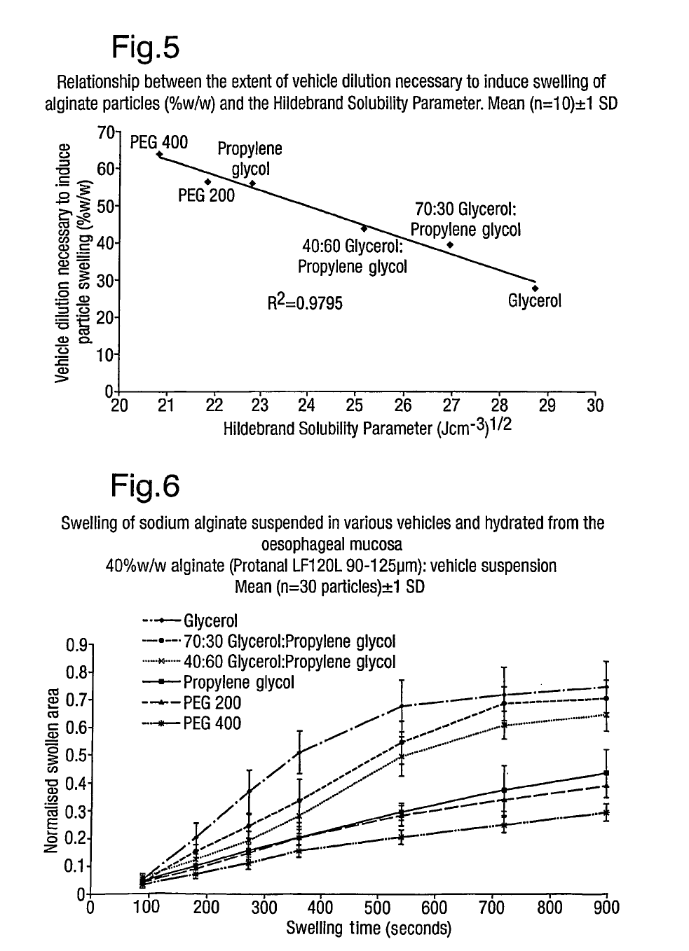

FIGS. 4 and 5 relate the Hildebrand solubility parameter to the modelled swelling rate at 80% w/w vehicle dilution and the extent of dilution necessary to indicate particle swelling.

### EXAMPLE SET 2

These examples examined the influence of the diluent choice on the swelling of suspended alginate particles when applied to oesophageal mucosa.

Based on the results of Examples Set 1 it is suggested that as a composition enters the upper gastrointestinal tract the suspended alginate will start to swell as the diluent is diluted by saliva present on the mucosal surface. Contact between the swelling alginate and mucosal surface would lead to the formation of a swollen bioadhesive film coating the tissue surface. The swelling of the suspended particles at the interface between mucosa and composition may be critical to the establishment of the bioadhesive layer.

To understand the influence of diluent choice on the swelling behaviour of alginate particles at the composition:mucosa interface it was desirable to be able to visualise the microstructure of the bioadhesive film as it developed on the mucosal surface.

Equipment

“Macroscope”—by which we mean a Cool Snap Pro Digital Camera attached to a Nikon AF Micro Nikkor 60 mm f/2.8D lens interfaced through a CoolSnap Pro PCI Interface card to a Pentium PIII 1 GHz PC. Image analysis was performed using Image ProPlus v4. (Media Cybernetics, Maryland, USA-Supplier Datacell Ltd, Finchampstead, Berkshire, UK) Thoma Haemocytometer Counting Chamber, Depth 0.1 mm, 0.0025 mm², Hawksley, England.

Methodology

Tissue Mucosa Preparation

Fresh porcine oesophagus was collected immediately after slaughter in phosphate buffered saline, and transported on ice. The musculature was removed by dissection within one hour of slaughter, leaving a clean epithelial tissue tube. A 25 mm×50 mm section of tissue was adhered to a microscope slide using cyanoacrylate glue (Super Glue®, Loctite (Ireland) Ltd), hydrated in 40 ml 0.9% w/v NaCl for 1 minute and washed in artificial saliva before being placed under the Macroscope.

Formulation Preparation

The following diluents were each prepared containing DMMB at 1.9 mM concentration.
Glycerol
Propylene glycol
PEG200
PEG400
70:30 w/w mix glycerol:propylene glycol
40:60 w/w mix glycerol:propylene glycol

Each solution was then used to prepare a 40% w/w suspension of sodium alginate. This was done by weighing out the appropriate amount of diluent and alginate and then mixing the two materials in a glass vial with a spatula.

Application of Formulation to Tissue Mucosa

The alginate suspension was applied to the tissue surface by filling the open chamber of the haemocytometer slide and inverting onto the tissue surface. The haemocytometer slide ensured a uniform, monolayer of suspension was spread over the tissue surface.

Image Capture and Analysis

The swelling of suspended alginate particles were visualised using the Macroscope. The Macroscope can be described as a macroscopic lens attached to a digital camera, interfaced to a PC enabling the capture of digital images visualising the micro-structure of the bioadhesive film. As the alginate particles hydrated on the mucosal surface and complexed with DMMB, the swollen area was delineated by the colour change from blue to purple. Image analysis software captured an image after a predetermined time period and converted a series of images into a movie depicting the extent of particle swelling over time. A digital grid was then placed over the entire image and image analysis performed on certain particles selected according to their specific grid reference. The measurement of the extent of radial particle swelling gave an insight into the characteristics of swollen alginate domain formation within the bioadhesive film.

The Macroscope showed that as the composition was placed on the mucosal surface the suspended alginate particles began to hydrate and swell. The presence of DMMB in the diluent meant that as the particles started swelling there was a colour change from blue to purple due to alginate:DMMB complexation. Using image analysis software it was possible to measure the change in the swollen area of suspended alginate particles over time.

FIG. 6 illustrates the change in the swollen area of alginate particles suspended in a range of diluents when placed on oesophageal mucosa.

The results in FIG. 6 suggest that the rate at which alginate particles swell when placed on oesophageal mucosa could be modulated by diluent choice. As is illustrated in the following table, the choice of diluent influenced both the rate of swelling between 0 and 360 seconds and the extent of swelling.

| | Rate of Swelling | Extent of Swelling |
|---|---|---|
| | (Change in | at t = 360 seconds |
| | normalised swollen | (Normalised |
| | area/time) Mean | swollen area) Mean |
| Diluent | (n = 30) ± 1SD | (n = 30) ± 1SD |
| Glycerol | 1.71 × 10⁻³ ± 3.14 × 10⁻⁴ | 0.5049 ± 0.0773 |
| 70:30 w/w | 1.07 × 10⁻³ ± 2.46 × 10⁻⁴ | 0.3299 ± 0.0776 |
| Glycerol:propylene | | |
| glycol | | |
| 40:60 w/w | 8.2 × 10⁻⁴ ± 8.2 × 10⁻⁴ | 0.2763 ± 0.0620 |
| Glycerol:propylene | | |
| glycol | | |
| Propylene glycol | 5.9 × 10⁻⁴ ± 1.16 × 10⁻⁴ | 0.2002 ± 0.0378 |
| PEG200 | 6.0 × 10⁻⁴ ± 6.50 × 10⁻⁵ | 0.1997 ± 0.0292 |
| PEG400 | 4.6 × 10⁻⁴ ± 6.57 × 10⁻⁵ | 0.1518 ± 0.0231 |

Rate and Extent of Swelling of Alginate Suspended in Diluent on Oesophageal Mucosa

Formulation-40% w/w alginate (Protanal LF120L 90-125 μm):diluent

Rate of swelling calculated between 90-360 seconds.

FIG. 6 illustrates that particles suspended in diluents containing glycerol swell most rapidly, and that different diluents exhibit differences in the equilibrium swollen area and the rate of swelling prior to equilibrium. For example, particles suspended in PEG 400 started to plateau at a normalised swollen area of 0.28 however particles suspended in glycerol were still swelling rapidly at a similar swollen area. Clearly diluent choice exerts an influence on particle swelling.

### EXAMPLE SET 3

These examples were carried out in order to explore the relationship between bioadhesion and swelling.

Equipment

Agilent UV-Visible System 8453 (Agilent Technologies UK Td, Stockport, England)

Erweka ZT44 USP/BP Disintegration Tester (Copley Instruments, Nottingham, England).

Methodology

Outline

The adhesion of the formulation was measured by everting a section of porcine oesophagus onto a plastic tube and attaching to a USP disintegration tester, a machine giving a vertical dipping motion, dipping the mucosa into a 40% w/w diluent:Protanal LF120L (90-125 μm) suspension. The tissue and adhered formulation were then washed in artificial saliva (as described above) by the vertical motion of the tester into a washing container. The container was replaced after a pre-determined time period relative to the extent of the total adhesion to provide approx 5 sample collections each with an analysable quantity of alginate (0.7 gL⁻¹) contained. After the formulation appeared to be totally detached (visual observation), the tissue was removed from the disintegration tester and agitated for 2 hours in artificial saliva to remove any residual adhered material. Following agitation, to ensure that all adhered alginate had been detached the mucosa was scraped and the residue analysed for alginate concentration.

Preparation of the Mucosal Surface

Fresh porcine oesophagus was collected immediately after slaughter in phosphate buffered saline, and transported on ice. The musculature was removed by dissection within one hour of slaughter, leaving a clean epithelial tissue tube. The tissue tube was then cut into 8 cm segments and the segments everted onto a disintegration tester rod.

The attached tissue was then rinsed gently and left to equilibrate for 1 minute in 0.9% sodium chloride.

Adhesion Testing

The tissue was attached to the disintegration tester and lowered into 16g of formulation (40% w/w alginate/diluent suspension) and left for 5 seconds. The disintegration tester was then started and the tissue and adhered alginate dipped in and out of 18ml artificial saliva (37° C.) at a rate and distance pre-determined by the USP. The dipping motion washed over the surface of the formulation-tissue and caused detachment of formulation via disintegration and dissolution.

Sample Collection

After a pre-determined time period, the disintegration tester was stopped and the artificial saliva washing container changed. The pre-determined time period was gauged from a preliminary experiment that provided an approximation of the washing time needed to detach the formulation. This time period was then divided to give a range of time frames during which an analysable quantity of composition (0.7 gL⁻¹ alginate) could have washed into the 18 ml of artificial saliva. The end-point, i.e. total detachment was determined by visual observation. Having reaching the end-point the tissue was removed from the disintegration tester and placed in 16 ml artificial saliva and left to stir for 2 hours. This was to ensure that residual adhered alginate was completely detached.

To validate total alginate detachment, the tissue was scraped and the scrapings dissolved in 1 ml artificial saliva and analysed for solubilised alginate. Analysable concentrations of alginate were never detected.

The total amount of alginate that had adhered to the section of oesophageal mucosal was therefore contained within all the washing containers. It was possible to sample each container and quantify the alginate present.

Quantification of Detached Sodium Alginate

Preparation of Alginate Standard Calibration Solutions

A 0.5% w/v sodium alginate solution was prepared in artificial saliva by stirring until completely dissolved. This was used to prepare 1 ml standard solutions over the range 0.7 to 3.0 gL⁻¹.

Preparation of Sample Solutions

1000 mg was sampled from each container, however if there was a high concentration of alginate it was necessary to dilute the sample to ensure the concentration of alginate to be analysed was within the assay range (0.7-3.0 gL⁻¹).

Alginate Assay Procedure

1 ml 0.8M sodium hydroxide was added to 1 ml alginate solution and neutralised after 5 minutes with 120 μL 2.25M citric acid. The samples were vortex mixed, 40 μL DMMB was added, remixed, incubated for 45 minutes at room temperature and the absorbance intensity measured at 520 and 650 nm in a 1 cm pathlength cell using an Agilent 8453 UV Spectrophotometer (Agilent Technologies UK Ltd, Stockport, England).

Since there is a linear relationship between the absorbance ratio 520:650 nm and alginate concentration within the range 0.7-3.0 gL⁻¹ it was possible to quantify the amount of alginate sampled from each container, therefore the level of detachment at each time point.

Having calculated the percentage retention at each stage of washing it was possible to determine the extent of retention for each formulation with increasing washing time and subsequently compare the bioadhesive characteristics.

Statistical Analysis

All statistical calculations were performed using GraphPad InStat v.3.05 (GraphPad Software Inc, San Diego, Calif., USA). one-way analysis of variance (ANOVA) and Tukey's multiple comparison test were undertaken at a significance level of p<0.05. Non-linear regression analysis was performed using GraphPad Prism v3.02 (GraphPad Software Inc, San Diego, Calif., USA).

Results and Discussion

Retention of Sodium Alginate Suspended in a Range of Water Miscible Diluents to Oesophageal Mucosa

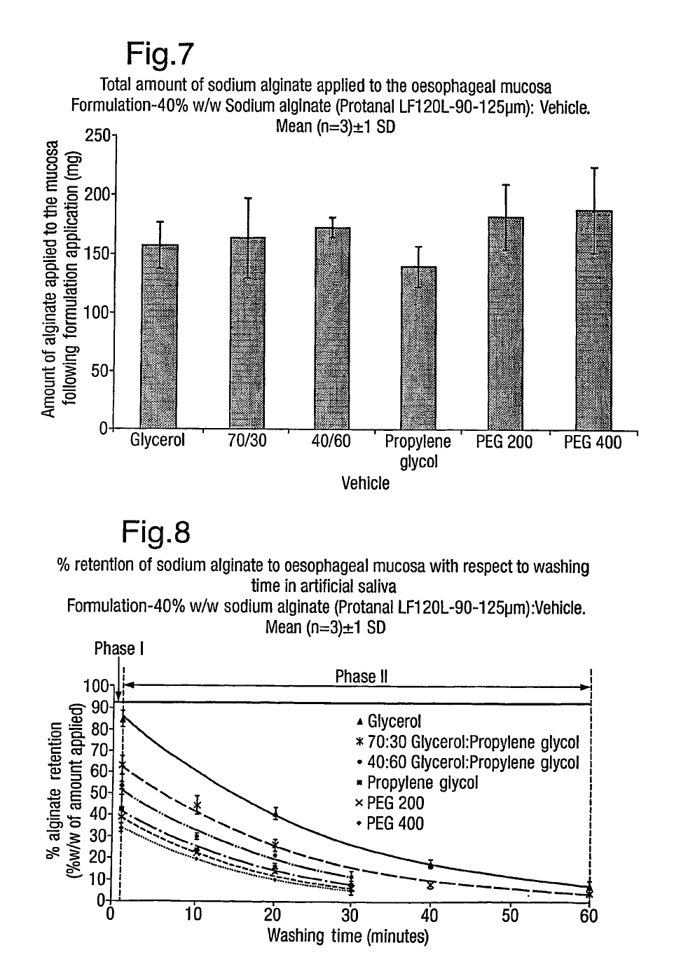

Following formulation application, FIG. 7 shows the influence of diluent choice on the total amount of alginate applied to the oesophageal mucosa. There was no significant different (p>0.05) between the individual diluents in the total amount of alginate applied to the mucosa.

Despite diluent choice not influencing the amount of alginate applied to the mucosa, the subsequent detachment of applied alginate from the mucosal surface during washing was diluent dependent. This is illustrated in FIG. 8.

FIG. 8 illustrates the retention of sodium alginate to oesophageal mucosa with increasing washing time. Retention was described as the % alginate retention, which was the % w/w of alginate still retained on the tissue at washing time X minutes, relative to the total amount of alginate applied to the tissue prior to washing at t=0.

It was clear the choice of diluent influenced the retention of alginate to the mucosal surface. Sodium alginate suspended in glycerol showed the greatest retention to the mucosal surface whereas alginate suspended in PEG400 was retained the least.

### EXAMPLE SET 4

It has been demonstrated by the foregoing that the swelling of alginate particles suspended in a water-miscible diluent influences the establishment of the bioadhesive interaction between alginate and mucosa. Particles suspended in a diluent that required the least dilution with artificial saliva to initiate swelling had the greatest retention to the mucosal surface. The recognition that the extent of diluent dilution with artificial saliva influenced particle swelling and subsequently alginate mucosal retention has implications for the in vivo performance of these formulations.

Following the administration of a dose of formulation to the patient, the suspended alginate would enter the oral cavity and be swallowed into the upper oesophagus. As the formulation migrates through the oral cavity and oesophagus the diluent would be diluted by saliva in the mouth and fluid on the mucosal surface. After sufficient diluent dilution, the suspended alginate particles would start to swell and be retained on the mucosa. Differences between diluents in the extent of diluent dilution necessary to initiate particle swelling may permit particle swelling to be activated to occur in a certain region of the gastro-intestinal tract governed by the relative ingress of saliva. In this manner, it may be possible to achieve site-specific retention of alginate. For example, alginate suspended in glycerol required the least dilution with artificial saliva to begin swelling and may be expected to become bioadhesive during the earlier stages of gastro-intestinal transit and be retained within the oral cavity or upper oesophagus.

Alternatively, PEG400 formulations may be transported into the lower oesophagus before they have reached a sufficient level of dilution to swell and adhere; thus adhesion would be delayed until reaching the lower oesophagus enabling delivery of the coating alginate layer to this site.

It was therefore considered important to understand how diluent choice may influence the regionalised distribution of retained alginate over the mucosal surface.

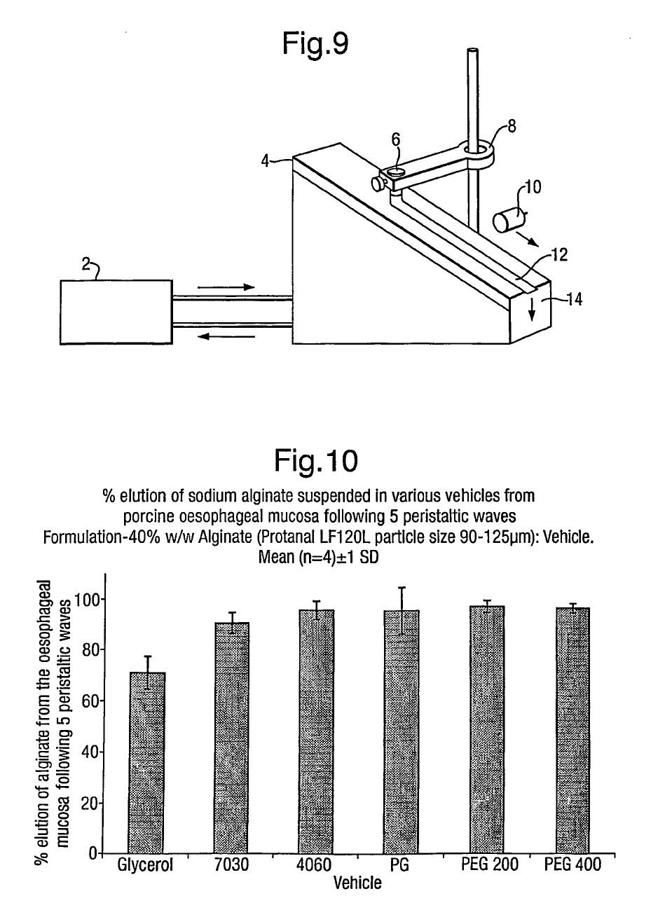

It was possible to characterise the distribution of retained alginate using an in-house in vitro bioadhesion test system. The “peristaltic tube” bioadhesion test system was specifically designed to measure the retention of potentially bioadhesive liquid formulations to the oesophagus. The “peristaltic tube” bioadhesion test system apparatus is shown below in FIG. 9. In FIG. 9 the following numerals refer to parts, as follows.
2—Water heater/circulator; supplies metal slope bed water jacket (37° C.)
4—Heated metal slope bed (45° slope)
6—Formulation and artificial saliva aliquots injected by syringe
8—Metal retort stand/clamp
10—Roller; provides peristalsis action
12—Oesophagus tube (covered with Clingfilm®)
14—Eluate fractions collected in vials

Retention of formulation within the oesophagus was determined by applying the formulation directly into the oesophageal tissue tube and washing through the oesophagus with repeated aliquots of artificial saliva. Following each wash the peristaltic action of swallowing was simulated using a roller.

After a series of washes and peristaltic waves, the oesophageal tissue tube was cut open and the distribution of retained alginate over the upper, mid and lower regions of the oesophageal tube calculated by scraping the mucosal surface and quantifying the concentration of alginate within the scrapings.

The aim in Example Set 4, using the “peristaltic tube” bioadhesion testing system, was to determine the influence of diluent choice on the distribution of retained alginate along the mucosa of the oesophageal tissue tube, in conditions mimicking peristalsis.

Preparation of Oesophagus

Fresh oesophagus was collected immediately after slaughter in phosphate buffered saline, and transported on ice. The musculature was removed by dissection within one hour of slaughter, leaving a clean epithelial tissue tube. The upper oesophagus was cut to ensure a uniform tube length of 31 cm.

Attachment of Oesophagus to Dosing Port

It was necessary to attach the oesophagus to a dosing port. The dosing port opened the oesophageal tube and provided a means of injecting formulation and artificial saliva directly into the oesophagus. The dosing port consisted of a plastic tube 32 mm long (internal diameter 8 mm) with two tightly fitting silicone rubber flanges attached. The flange end of the dosing port was inserted into the top end of the oesophagus which was secured tightly between the two flanges using a cable tie.

Mounting of Tissue onto Slope

The dosing port and oesophagus were attached into a retort stand and the tissue mounted onto a 40 cm long×6 cm wide aluminium slope (45 degrees to horizontal). The oesophagus was positioned so the lower end extended beyond the bottom of the slope leaving the lower oesophageal aperture free for elution into a collection vessel. The whole length of the oesophageal tube was covered with Clingfilm®, to provide insulation and prevent moisture loss. Using a water flow heater, water was circulated through the underside of the slope to heat the metal slope surface to a temperature of 37° C.±1° C. The tissue was left positioned over the heated slope and allowed to equilibrate to 37° C.

Prewashing the Oesophagus

It was necessary to prewash the oesophagus prior to the administration of formulation. This was to ensure that any food retained within the oesophagus and mucus present on the oesophageal mucosa was washed away. The oesophagus was washed by injecting ten 10 ml aliquots of artificial saliva at 37° C. through the dosing port. After each injection the plastic roller was run down the length of the tissue tube, using a light pressure of 100-150 g, to elute the liquid by peristalsis.

Administration of Formulation into the Oesophagus

The formulation was administered into the oesophagus using a dosing syringe assembly. The dosing assembly consisted of a 10 ml luer lock syringe tightly fitted into the top of a 1 ml syringe body via an adapter made from the cap of the 10 ml luer lock syringe.

The dosing assembly was filled by filling the 1 ml syringe with formulation and then separately weighing 10 g of formulation into the 10 ml syringe taking care to wipe any excess product from the outside of the syringe. The two syringes were then fitted together and re-weighed. The dosing syringe was inserted into the dosing port so that the upper end of the dosing port was in contact with the top finger bar of 1 ml syringe. This ensured the dosing assembly penetrated 53 mm below the lower end of the dosing port and standardised the position within the oesophagus of formulation application. The upper 10 ml syringe was slowly discharged, it was necessary to pinch the oesophagus tube around the inserted 1 ml syringe to prevent backflushing of dosed formulation. The dose assembly was slowly withdrawn from the oesophagus and reweighed to calculate the weight of formulation applied into the oesophagus.

Elution of Formulation from the Oesophagus

The formulation was eluted from the oesophageal tube using a combination of washing with artificial saliva and reproducing peristaltic waves using the plastic roller.

Immediately following formulation administration and prior to washing 5 peristaltic waves were initiated down the length of the oesophagus using the roller to elute excess formulation. It was possible to quantify the initial detachment of alginate following 5 peristaltic waves by analysing the alginate concentration of the eluent using the DMMB complexation assay.

The remaining alginate retained on the mucosal surface was then eluted by injecting 30 1 ml aliquots of artificial saliva at 37° C. through the dosing port and after each 1 ml wash recreating a peristaltic wave using the roller. The artificial saliva wash was injected through the dosing port using a washing syringe. The washing syringe was 1 ml plastic syringe fitted with a flange to restrict its penetration below the dosing port to 14 mm. This ensured the washing started 40 mm above the point of application of the formulation and prevented the accumulation of a reservoir of uneluted formulation around the dosing port.

Measurement of Alginate Retained on the Mucosal Surface

Following 30 1 ml washes the oesophageal tube was removed from the slope and divided into 3 sections of 70 mm representing the top, middle and lower portion of the oesophagus. Each section was cut open lengthwise to expose the inner mucosal surface and stretched out flat on a polystyrene support. The tissue was scraped using a glass microscope slide to remove retained alginate from the mucosal surface.

The scrapings were washed from the slide into a beaker and diluted with artificial saliva to an approximate alginate concentration of 0.7-2.5 gL⁻¹. The scrapings were left to stir overnight to ensure complete dissolution of the alginate. It was possible to quantify the amount of alginate scraped from each tissue section using the DMMB complexation assay. The percentage retention of alginate relative to the dose applied could then be calculated.

Formulations Tested

A 40% w/w formulation of sodium alginate (Protanal LF120L), particle size 90-125 μm, was suspended in the following diluents by thoroughly mixing the two phases with a spatula.

Diluents used—glycerol; 70:30 w/w glycerol:propylene glycol; 40:60 w/w glycerol:propylene glycol; propylene glycol, PEG 200; PEG 400.

Statistical calculations were undertaken using GraphPad InStat v.3.00 (GraphPad Software Inc, San Diego, Calif., USA). One-way analysis of variance (ANOVA) and t-tests and were undertaken at a significance level of p>0.05.

Results and Discussion

Elution of Alginate Following 5 Peristaltic Waves

Using the “peristaltic tube” bioadhesion testing system it was possible to investigate the influence of diluent choice on alginate retention within the oesophagus. FIG. 10 illustrates the % of the total amount of alginate applied to the oesophageal mucosa that was eluted from the oesophagus following five initial peristaltic waves prior to washing.

It is clear that suspending alginate in glycerol significantly (p<0.05) reduced the elution of alginate from the oesophagus following dosing. It would appear that alginate suspended in glycerol rapidly established a bioadhesive interaction with the oesophageal mucosa and was able to resist the disruptive effect of 5 peristaltic waves. The ability of alginate suspended in glycerol to rapidly establish a bioadhesive interaction with the tissue surface is analogous to the retentive behaviour described above after 60 seconds of washing. The increased retention of the glycerol based formulation may be explained by the increased propensity of alginate particles to swell in glycerol when hydrated within the oesophagus and form adhesive and cohesive interactions.

Mucosal Retention of Alginate Following Washing

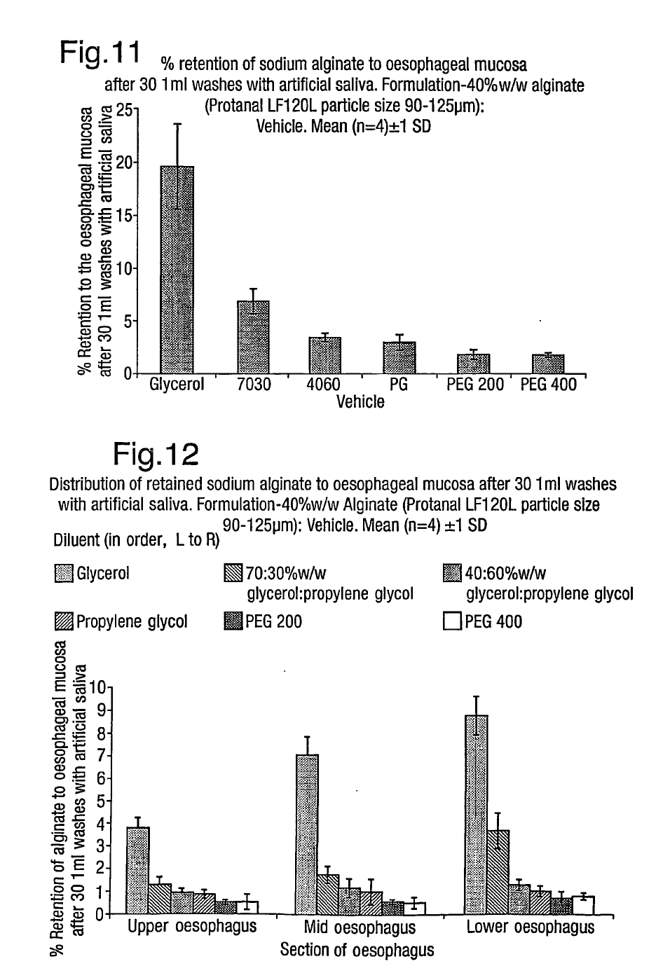

Following application of the formulation to the mucosa the oesophagus was washed with 30 1 ml washes of artificial saliva. FIG. 11 shows the % of the total amount of alginate dosed into the oesophagus that was still retained on the musocal surface after washing.

Sodium alginate suspended in glycerol and 70/30 w/w glycerol:propylene glycol had a significantly (p<0.05) greater retention on the oesophageal mucosa than alginate suspended in any other diluent. The increased retention of alginate suspended in these two diluents after washing was related to there being more retained after the initial 5 peristaltic waves (FIG. 10). Alginate had a greater propensity to swell when suspended in glycerol and 70:30 w/w glycerol:propylene glycol compared to the other diluents.

The increased ability to swell was responsible for more alginate being retained in the oesophagus following 5 peristaltic waves and ultimately more being retained after washing. However for alginate suspended in. 40:60 w/w glycerol:propylene glycol, propylene glycol, PEG200 and PEG400 in excess of 95% of the applied dose was eluted following 5 peristaltic washes. Alginate suspended in each of these diluents was incapable of swelling sufficiently during transit through the oesophagus and was unable to establish a bioadhesive interaction with the mucosa. Consequently after 30 1 ml washes there was a very small amount (<3% of the applied does) retained within the oesophagus.

It would appear that the ability of suspended alginate to swell during transit through the oesophagus was a determining factor influencing the extent of alginate retention following washing.

Having described the total amount of retention to the oesophagus after 30 1 ml washes, it was considered important to understand how the retained alginate was distributed over the mucosal surface. Different formulations may be retained in different regions of the oesophagus which may be critical to the clinical efficacy of a potential mucoprotective formulation. Within the scope of this work the ideal formulation would have swollen sufficiently during gastro-intestinal transit to be retained on the mucosal surface of the lower oesophagus and provide a barrier against gastric refluxate.

FIG. 12 demonstrates how diluent choice influenced the retention of alginate in 3 regions of the oesophagus, the upper, mid and lower section.

In each region of the oesophagus alginate suspended in glycerol was retained to a significantly (p<0.05) greater extent than in any other diluent. Similarly in the lower oesophagus the formulation based on 70:30 w/w glycerol:propylene glycol was retained significantly more than all the other diluents except glycerol.

It was also demonstrated that for alginate suspended in either glycerol or 70:30 w/w glycerol: propylene glycol significantly (p<0.05) more alginate was retained in the lower oesophagus than in the upper region. The increased retention of alginate within the lower region of the oesophagus may be related to formulation hydration. As the formulation is injected into the oesophagus the bolus injection migrates down the oesophagus due to the initial peristaltic waves. Having reached the lower oesophagus the suspended alginate will be in the most swollen state due to the increased dilution by fluid in the oesophagus. The presence of a relatively greater amount of swollen alginate in the lower oesophagus facilitates the formation of adhesive and cohesive interactions and may explain the greater retention of alginate within this region.

It has been shown in FIG. 11 that alginate suspended in diluents other than glycerol and 70:30 glycerol:propylene glycol had the lowest extent of retention after washing. The low mucosal retention of alginate suspended in these diluents has been attributed to the inability of alginate to swell and form the necessary bioadhesive interactions with the mucosa. Additionally FIG. 12 demonstrated there were no significant differences (p>0.05) in the amount of alginate retained in each region of the oesophagus. The even distribution of retained alginate within the oesophagus suggests that even in the lower oesophagus the alginate was incapable of swelling and being retained to a greater degree than in the upper oesophagus. This suggests that alginate suspended in 40:60 w/w glycerol:propylene glycol, propylene glycol, PEG 200 and PEG 400 passed through the whole length of the oesophagus in a relatively unswollen state and was unable to be sufficiently diluted to swell and be retained at this site. However, it should be kept in mind that retention using these other vehicles may be substantially improved if they are diluted with water before application to an oesophagus.

Conclusions

It has been possible using diluent choice to alter the distribution of alginate retention within the oesophagus. This has been discussed in relation to the ability of alginate to swell within the oesophagus. Alginate suspended in glycerol was most capable of swelling following administration into the oesophagus and consequently was able to form sufficient cohesive and adhesive interactions to be retained throughout the oesophagus. Retention was greatest in the lower oesophagus. We believe this is due to the highly swollen state of the alginate at this particular point of transit.

### CONCLUSION FROM EXAMPLES SETS 1-4

It has been demonstrated that it is possible to modulate both the swelling and the retention to oesophageal mucosa of alginate particles suspended in a water-miscible diluent. Modulation has been achieved by the choice of diluent which appears to control:
The amount of formulation dilution necessary to trigger particle swelling
The rate of particle swelling on mucosal tissue
The initial and duration of retention of formulation to the mucosal surface
Distribution of retention of alginate on the oesophageal mucosa

Formulations based on the delivery of dry sodium alginate powder in a variety of water-miscible diluents enable control over the development of bioadhesion as a function of formulation hydration. This phenomenon may offer a means of targeting the oesophagus (in these examples) and other bodily surface as a site of adhesion (in other examples). The ability to design a formulation that could resist swelling until a sufficient level of dilution has occurred to trigger adhesion to a bodily surface would be highly desirable.

The ideal composition for oesophageal retention would not swell in the mouth and would migrate along the oesophageal wall as a bolus under the influence of normal peristalsis and GI transit. Having reached the lower oesophagus the formulation would start to swell and develop the necessary adhesive and cohesive properties to form a bioadhesive film within the lower oesophagus. If the bioadhesive barrier was of sufficient integrity it would resist. dissolution and disintegration due to the washing effects of saliva and peristalsis and maintain a protective coat over the mucosal surface.

A composition of this type would provide an excellent means of treating/preventing oesophageal tissue damage due to gastric reflux.

Compositions of the type described above could also provide a means of providing delayed release of other active ingredients to other parts of the gastro-intestinal tract.

Compositions of the type described above could also be useful in non-therapeutic applications.

1.-26. (canceled)

27. A liquid composition for adherence to a surface, which composition comprises from 20 to 60 wt % water-swellable polymer particles suspended in a water-miscible liquid diluent, wherein
the liquid diluent is free of water or includes an amount of water insufficient to fully swell the polymer particles,
the polymer particles do not include both anionic particles and cationic particles, and
the composition does not comprise an additional pharmaceutically active agent.

28. A composition according to claim 27 which is intended for treating or preventing inflammation of, damage to, or disease in a bodily surface, and in which the liquid diluent is pharmaceutically acceptable.

29. A composition according to claim 27 wherein the polymer particles comprise an alginate.

30. A composition according to claim 29 wherein the alginate is sodium alginate.

31. A composition according to claim 27 wherein the polymer particles have a mean particle size of from 30 to 500 μm.

32. A composition according to claim 27 wherein the liquid diluent comprises one or more materials selected from the groups consisting of a monohydric alcohol, a polyhydric alcohol, a sugar alcohol and a sugar polyol.

33. A composition according to claim 32 wherein the liquid diluent comprises a polyhydric alcohol.

34. A composition according to claim 33 wherein the liquid diluent comprises one or more materials selected from the group consisting of glycerol, a glycol, a glycol ether and a polyalkylene glycol.

35. A composition according to claim 34 wherein the liquid diluent is glycerol.

36. A composition according to claim 34 wherein the liquid diluent comprises X parts of propylene glycol admixed with (100 minus X) parts of glycerol (w/w), where X is a number up to 30.

37. A composition according to claim 27 wherein the liquid diluent has a Hildebrand Solubility Parameter not greater than 35 (Jcm⁻³)^{1/2}.

38. In the manufacture of a liquid pharmaceutical composition for treating or preventing inflammation of or damage to a bodily surface, the improvement which comprises incorporating into said pharmaceutical composition a surface-adhering composition comprising from 20 to 60 wt % of water-swellable polymer particles suspended in a pharmaceutically acceptable water-miscible liquid diluent, the liquid diluent being free of water or containing an amount of water insufficient to fully swell the polymer particles, wherein the polymer particles do not comprise both anionic particles and cationic particles and wherein the adhering composition does not comprise any additional pharmaceutical agent.

39. A method of treating a surface comprising applying to the surface a liquid composition comprising water-swellable polymer particles suspended in a water-miscible liquid diluent in which the polymer particles do not comprise both anionic particles and cationic particles and the composition is free of water or includes an amount of water insufficient to fully swell the polymer particles, wherein, prior to application of the composition to the surface or during application of the composition to the surface, water is mixed with the composition to cause swelling of the polymer particles in the vicinity of the surface, an increase in the viscosity of the composition and the formation of an adhered coating of the polymer on the surface.

40. A method according to claim 39 wherein the composition is intended for treating or preventing inflammation of, damage to, or disease in a bodily surface and the liquid diluent is pharmaceutically acceptable.

41. A method according to claim 40 in which the composition comprises from 20 to 60 wt % of said polymer particles.
